(19) United States
(12) Patent Application Publication
(10) Pub. No.: US 2022/0252580 A1
(43) Pub. Date: Aug. 11, 2022

(54) REVERSIBLE CELL DETECTION WITH CONJUGATES HAVING A LINKER FOR INCREASED FLUORESCENT BRIGHTNESS AND AN ENZYMMATICALLY RELEASABLE FLUORESCENT MOIETY

(71) Applicant: Miltenyi Biotec B.V. & Co. KG, Bergisch Gladbach (DE)

(72) Inventors: Christian DOSE, Bergisch Gladbach (DE); Jennifer PANKRATZ, Bergisch Gladbach (DE)

(73) Assignee: Miltenyi Biotec B.V. & Co. KG, Bergisch Gladbach (DE)

(21) Appl. No.: 17/603,598

(22) PCT Filed: Apr. 23, 2019
(86) PCT No.: PCT/EP2019/060403

§ 371 Date: Oct. 14, 2021

(51) Int. Cl. G01N 33/533; G01N 1/30

(52) U.S. Cl. CPC G01N 33/533; G01N 1/30

(57) ABSTRACT

The invention is directed to a conjugate for labelling a target moiety on a cell, characterized with the general formula (I) (Xₒ-L)ₙ-P-Yₘ, with Y: antigen recognizing moiety recognizing the target moiety, P: enzymatically degradable spacer, X: fluorescent moiety, L: linker unit comprising one or more polyethyleneglycol residues n, m: integer between 1 and 100, o integer between 1 and 100 wherein L covalent bounds the fluorescent moiety X and the enzymatically degradable spacer P and Y is covalently bound to the enzymatically degradable spacer P and wherein the enzymatically degradable spacer P is selected from the group consisting of polysaccharides, polyesters, nucleic acids, and derivatives thereof. Method of detecting a target moiety in a sample of biological specimen with the conjugate.

### BACKGROUND

The present invention is directed to a process for detection of a target moiety in a sample of biological specimens by labelling the target moiety with a conjugate having an antigen recognizing moiety and a fluorescent moiety connected via enzymatically degradable spacer and a hydrophilic linker group comprising polyethylene glycol, wherein after detecting or isolating the target moiety, the degradable spacer is enzymatically degraded, thereby releasing the target cells from at least the fluorescent moiety.

Immunofluorescent and immunomagnetic labelling are important for the detailed analysis and specific isolation of target cells from a biological specimen in both research and clinical applications. The techniques combine the specific labelling of a target moiety with conjugates having a detectable unit like a magnetic particle to retain and therefore isolate cells in a magnetic field, or like a fluorescent dye or transition metal isotope mass tag to detect and characterize cells by microscopy or cytometry. For immunofluorescence analysis, a vast number of variants in view of antibodies, fluorescent dyes, flow cytometers, flow sorters, and fluorescence microscopes has been developed in the last two decades to enable specific detection and isolation of target cells. One issue in immunofluorescence technology is the detection threshold and brightness of the fluorescence emission, which can be enhanced, for example, by better detectors, filter systems, lasers, or modified fluorescent dyes i.e. with better quantum yield. Immunofluorescent conjugates typically comprise multiple dyes to increase the fluorescence intensity but brightness is limited by self-quenching mechanism caused by dimer, trimer or multimer formation.

Recently, the flexibility regarding downstream applications and sequential detection or isolation cycles for various applications as magnetic cell enrichment, flow sorting or fluorescence microscopy evolved by the development of reversible labelling techniques. Those techniques allow for the removal of the fluorescent or magnetic labelling after cell sorting or cell analysis. Especially for technologies based on sequentially cycles of labelling-detection-elimination with high multiplexing potential to map, e.g., protein networks, the elimination of the fluorescence signal is essential. However, these technologies are based on oxidative destruction of conjugated fluorescent moieties by photo- or chemical bleaching procedures (U.S. Pat. No. 7,741,045 B2, EP 0810 428 B1 or DE10143757) and are subjected to steric hindrances by antibodies remaining on the specimen

In this respect in the last years several approaches for bright immunofluorescent conjugates and for reversible labelling with immunoconjugates were developed.

For example, it is known to use PEG as a linker to reduce fluorescence quenching as disclosed by Y. Guo et al., J. Am. Chem. Soc. 2012, 134, 19338-19341. Here, the use of PEG as a linker to suppress troublesome interaction of the fluorochrome with biomolecules and improve quantum yield is described. However, there is no indication of use of PEG in multimerization. Each fluorochrome is linked to a RGD peptide via said PEG linker.

EP3098269 A1 teaches multimerization of fluorochromes on branched polyether scaffolds. A core moiety of 20 to 200 atoms serves as a tethering place for multiple PEG linkers carrying fluorochromes at the other end of the linker chain. The multimerized polyether scaffolds can be conjugated to antibodies. The polyether scaffold prevents quenching and unspecific binding of the fluorochromes. However, this publication does not teach any methods of reversible labelling or release of label. The core moiety is too small to allow for enzymatic degradation of the polyether scaffold and monomerization of the fluorochromes. Therefore, EP3098269 A1 is directed at providing a bright fluorescent label by multimerization of unquenched fluorochromes, but does not disclose a method of releasing said label.

WO 96/31776 describes a method to release after separation magnetic particles from target cells by enzymatically cleaving a moiety of the particle coating, or a moiety present in the linkage group between the coating and the antigen recognizing moiety. An example is the application of magnetic particles coated with dextran and/or linked via dextran to the antigen recognizing moiety. Subsequent cleavage of the isolated target cells from the magnetic particle is initiated by the addition of the dextran-degrading enzyme dextranase. Therefore, WO 96/31776 is directed to release a magnetic label from a target moiety by enzymatic digestion, but does not disclose a method a fluorescent label.

A similar method is disclosed in EP3037821, with the detection and separation of a target moiety according to, e.g. a fluorescence signal, with conjugates having an enzymatically-degradable spacer for reversible fluorescent labelling.

An embodiment of EP3037821 is directed to a covalent multimerization strategy for low-affinity antigen recognizing moieties. The strategy provides low-affinity antigen recognizing moieties and a detection moiety, e.g. fluorescent dye, which are covalently linked and therefore covalently multimerized via an enzymatically degradable spacer. The covalent linkage enables a stable and defined multimerization and the option for multiple parameter labelling. During the enzymatic degradation of the spacer the detection moiety is released and the low-affinity antigen recognizing moiety is monomerized. Therefore, EP3037821 is directed to release a fluorescent label from a target moiety by enzymatic digestion and discloses a method for reversible covalent multimerization of low affinity antigen recognizing moieties, but does not provide a method to prevent fluorescent quenching or enhance fluorescent brightness though preserving releasability.

U.S. Pat. No. 5,719,031 describes dextran-fluorochrome-conjugates, wherein the degree of labelling is high enough to furnish fluorescent quenching. Therefore, degradation is accompanied by an enhancement of fluorescence emission signal, which is used for the quantification of the enzymatic digestion process. Therefore, U.S. Pat. No. 5,719,031 discloses a method wherein fluorescence quenching of the in the dextran-fluorochrome conjugates is desired and not prevented.

Fluorescence quenching is also described in GB2372256. Cells are stained with a conjugate comprising a plurality of fluorescent dyes attached via a linker to an antibody. Since the high density of fluorescent dyes will quench the fluorescence signals, GB2372256 describes an enzymatic degradation of the linker in order to release fluorescent dyes from the conjugate. The released fluorescent dyes are not subject to self-quenching, resulting in more intense fluorescence signals, i.e. in better resolution. However, since the fluorescence signals are detected after release from the target, the identification of target moieties on the cell surface is not possible with the method according to GB2372256. Furthermore, it is not possible to detect more than one target simultaneously, since the resulting mix of fluorescence signals cannot be assigned to a specific conjugate and/or target.

U.S. Pat. No. 9,023,604 discloses a method of reversible labelling based on indirect, non-covalent labelling of receptor molecules on target cells with reversible multimers. Receptor binding reagents characterized by a dissociation rate constant about 0,5×10−4 sec-1 or greater with a binding partner C are multimerized by a multimerization reagent with at least two binding sites Z interacting reversibly, non-covalently with the binding partner C to provide complexes with high avidity for the target antigen. The detectable label is bound to the multivalent binding complex. Reversibility of multimerization is initiated upon disruption of the binding between binding partner C and the binding site Z of the multimerization reagent. An example for the strategy are multimers of Fab-StreptagII/Streptactin wherein the multimerization can be reversed by the competitor Biotin. Therefore, U.S. Pat. No. 9,023,604 discloses a method for reversible non-covalent multimerization of low affinity antigen recognizing moieties, but is silent on strategies for reversible covalent multimerization and multiple parameter labelling or strategies to enhance fluorescent brightness or preserve releasability.

As mentioned EP3037821 describes conjugates with the general formula Xn-P-Ym consisting of detection moieties X, an enzymatically degradable spacer P and antigen recognizing moieties Y, that enable multiple parameter fluorescent labelling and cleaving of the detection moiety by enzymatically degradation of the spacer P.

A different approach is taken by WO2007109364, wherein releasable conjugates are disclosed with quenched fluorescent dyes when bound to a target. The conjugated contain a “protease cleavage site”, i.e. a spacer unit only degradable by a protease enzyme. After digesting the “protease cleavage site”, the fluorescent dyes are free to emit radiation for detection purposes. This approach is intended for indirect detection of cells and not for localization of targets on a cell surface.

The challenge in the development of these immunofluorescent conjugates for reversible labelling is to ensure the maximum fluorescence brightness and high reversibility. Theoretically, the increase of the degree of labelling with detection moieties on the enzymatically degradable spacer P enhances the fluorescence emission intensity. But the development revealed two limiting factors as an increased degree of labelling and proximity of fluorescent dyes furnished fluorescent quenching and therefore decreased fluorescence intensity, and the reduction of enzymatically cleavage efficiency. That is, increasing the amount of fluorescent labelling does not lead to a proportional increase of fluorescence signal intensity and furthermore decreases the enzymatic release by sterically hampering the access of the enzyme to the substrate.

### SUMMARY

It was therefore an object of the invention to provide a conjugate and a method for specific labelling, detection and de-labelling of target moieties in a sample of biological specimen in order to enable further labelling, which avoids fluorescence quenching.

Surprisingly, it was found that the implementation of a PEG-linker between the enzymatically degradable unit P and the fluorescent moiety X preserves the fluorescence of the fluorescent moiety which is otherwise lost by quenching, allowing the use of a lower degree of labelling, which in turn improves release by enzymatical cleaving.

It should be noted that the conjugates according to the invention emit fluorescent radiation when bound or even when not bound to a target cell, i.e. do not show the with quenched fluorescent as the dyes disclosed in WO2007109364. Without being bound to this theory, the quenched fluorescent might origin from the dendrimers used in WO2007109364, which sterically hamper the excitation/emission process. After separating from the dendrimer by enzymatic degradation of the spacer, the fluorescence capability of the dyes is restored. Since “quenched fluoresce” does not occur in the present conjugates, the conjugates according to WO2007109364 are chemically different from conjugates of the present invention.

Accordingly, the invention is directed to a conjugate for labelling a target moiety on a cell, characterized with the general formula

(Xₒ-L)ₙ-P-Yₘ, (I)
with Y: antigen recognizing moiety recognizing the target moiety,
   P: enzymatically degradable spacer,
   X: fluorescent moiety,
   L: linker unit comprising one or more polyethylene glycol residues
   n, m: integer between 1 and 100,
   o: integer between 1 and 100
wherein L covalent bounds the fluorescent moiety X and the enzymatically degradable spacer P and Y is covalently bound to the enzymatically degradable spacer P and wherein the enzymatically degradable spacer P is selected from the group consisting of polysaccharides, polyesters, nucleic acids, and derivatives thereof.

The conjugates utilized in the invention may for example have the general sequence “fluorescent dye(X)-PEG(L)-Dextran(P)-antibody(Y)” or “fluorescent dye(X)-PEG(L)-Dextran(P)-Fab(Y)”. Specific conjugates thereof are described in the examples.

The conjugates of the invention show an increased fluorescence intensity implemented by the linker L as compared to conjugates of the prior art and are suitable for multiple parameter labelling to target more than one target moiety in the sample of biological specimen. Since the fluorescent moiety of the conjugate can be removed from the target cells by addition of an enzyme, re-labelling of the cells with different antigen recognizing moieties carrying the same fluorescent moiety is possible, which provides additional possibilities for cell analysis or isolation. Compared to prior art technologies the present method enables a fast and less invasive protocol and avoids the implementation of reactive oxygen species, high energy or heat which may be harmful for the object of interest.

Furthermore, object of the invention is a method for detecting a target moiety in a sample of biological specimen by:
a) providing at least one conjugate having the general formula I

(Xₒ-L)ₙ-P-Yₘ (I)
with Y: antigen recognizing moiety recognizing the target moiety,
   P: enzymatically degradable spacer,
   X: fluorescent moiety,
   L: linker unit comprising one or more polyethylene glycol residues
   n, m: integer between 1 and 100,
   o integer between 1 and 100
wherein L covalent bounds the fluorescent moiety X and the enzymatically degradable spacer P and Y is covalently bound to the enzymatically degradable spacer P.
b) contacting the sample of biological specimens with the conjugate according to formula (I), thereby labelling the target moiety recognized by the antigen recognizing moiety Y
c) detecting the target moiety labelled with the conjugate with the fluorescent moiety X.

### BRIEF DESCRIPTION OF THE DRAWINGS

FIG. 1 shows schematically the method of the invention by specific labelling and release of a target moiety on a cell as biological specimen with conjugates of high-affinity (a) or low-affinity (b) antigen recognizing moiety Y, enzymatically degradable spacer P, and fluorescent moiety X conjugated via a linker unit L to the enzymatically degradable spacer P.

FIG. 2 shows exemplary results of absorption and fluorescence emission of dextran-PEG-coumarin-dye and dextran-coumarin-dye with different degrees of labeling at constant concentration of dextran.

FIG. 3 shows exemplary histograms of the result of flow cytometry analysis of the single parameter labeling with different anti-CD4-Fab-dextran-PEG-coumarin-dye conjugates (a-c) according to the invention in comparison to anti-CD4-Fab-dextran-coumarin-dye conjugate (d).

### DETAILED DESCRIPTION

The method and the conjugate of the invention are preferable used for in vitro detection of target cells.

For the purpose of the present invention, covalent bonds are defined as bonds between atoms sharing electron pairs or quasi-covalent bonds between non-covalent interaction partners with an equilibrium dissociation constant of less than 10E-9 M. Non-covalent bonds are defined as bonds with an equilibrium dissociation constant of greater than 10E-9 M.

The method of the invention may involve the removal of the antigen recognizing moiety Y from the target moiety. The method may therefore involve a step d) wherein the enzymatically degradable spacer P is degraded by an enzyme, thereby cleaving the fluorescent moieties X from the labelled target moiety.

In this respect, the invention encompasses two embodiments by using conjugates with high-affinity (a) or low-affinity (b) antigen recognizing moieties Y.

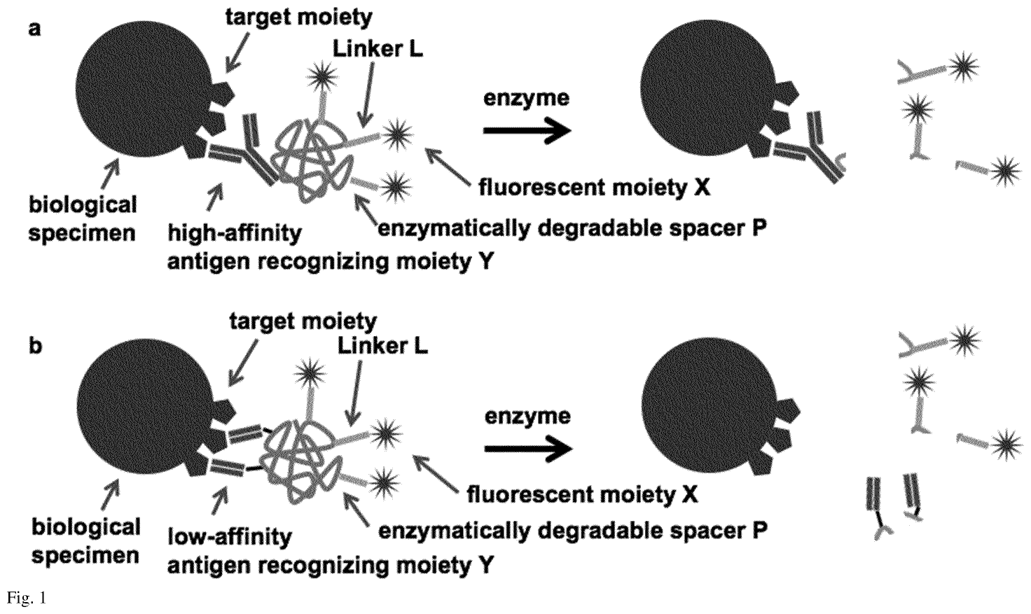

FIG. 1 shows schematically these embodiments of the invention by specific labelling of a target moiety on a target cell as biological specimen with conjugates of high-affinity (a) or low-affinity (b) antigen recognizing moiety Y, enzymatically degradable spacer P, linker unit L and fluorescent moiety X.

A high-affinity antigen recognizing moiety Y binds stable to a target moiety in a 1:1 ratio, i.e. n=1 in formula (I). When the spacer is enzymatically degraded, a high-affinity antigen recognizing moiety provide a stable bond which results in the removal of the fluorescent moiety X, the linker moiety L and the spacer P.

In a variant of the method according to the invention in step d), the enzymatically degradable spacer P is degraded by an enzyme, thereby cleaving the fluorescent moieties from X and the antigen recognizing moieties Y from the labelled target moiety.

This can be achieved by providing the conjugates with low-affinity antigen recognizing moieties Y. Such low-affinity antigen recognizing moieties do not provide a stable binding to the target moiety in a 1:1 ratio, but several low-affinity antigens recognizing moieties can be multimerized in one conjugate and therefore bind to the target moiety, i.e. n>1 in formula (I). Low-affinity antigen recognizing moieties will be monomerized during the degradation. Therefore, after dissociation of the monomerized low-affinity antigen recognizing moieties the target moiety is removed from the fluorescent moiety X, the linker moiety L, the spacer P and the antigen recognizing moiety Y. The stability of a non-covalent bond can be described by the equilibrium dissociation constant (KD), the dissociation rate constant (k(off)) and the association rate constant (k(on)) wherein KD=k(off)/k(on). Low-affinity antigen recognizing moieties can be characterized by the range of the equilibrium dissociation constant (KD) is equal or greater than 0.5E-08 M and the range for dissociation rate constant (k(off)) is equal or greater than 1E-03 sec-1, preferentially, the range for the equilibrium dissociation constant (KD) is between 0.5E-08 M and 1E-04 M and the range for dissociation rate constant (k(off)) is between 1E-03 sec-1 and 1E-00 sec-1.

In further embodiments of the invention, the enzymatically degradable spacer P is further provided with at least one covalent bound linker unit L not bound to a fluorescent moiety X and/or with at least one covalent bound fluorescent moiety X not bound to a linker unit L according to general formula (Xₒ-L)ₙ-P(L)ₗ(X)ₓ-Yₘ. wherein 1 and x are integer between 0 and 100 and n,o,m have the meaning as already disclosed.

In other words, it is possible that one or more fluorescent moieties X are be coupled without a linker L to the enzymatically degradable spacer P and/or that one or more linker L are be coupled without a fluorescent moiety X to the enzymatically degradable spacer P, both variants with the proviso that at least one (Xₒ-L) unit is covalently bound to the enzymatically degradable spacer P

For example, the conjugate may have the general formula (Xₒ-L)ₙ-P(L)ₗ-Yₘ. with 1 as integer in the range of 1-100 or (Xₒ-L)ₙ-P(X)ₓ-Yₘ with x as integer in the range of 1-100 or (Xₒ-L)ₙ-P(L)ₗ(X)ₓ-Yₘ with 1 and m as integer in the range of 1-100.

### Target Moiety

The target moiety to be detected with the method of the invention can be on any biological specimen, like tissues slices, cell aggregates, suspension cells, or adherent cells. The cells may be living or dead. Preferable, target moieties are antigens expressed intracellular or extracellular on biological specimen like whole animals, organs, tissues slices, cell aggregates, or single cells of invertebrates, (e.g., Caenorhabditis elegans, Drosophila melanogaster), vertebrates (e.g., Danio rerio, Xenopus laevis) and mammalians (e.g., Mus musculus, Homo sapiens).

### Fluorescent Moiety

Suitable fluorescent moieties X are those known from the art of immunofluorescence technologies, e.g., flow cytometry or fluorescence microscopy. In these embodiments of the invention, the target moiety labelled with the conjugate is detected by exciting the fluorescent moiety X and detecting the resulting emission (photoluminescence). Useful fluorescent moieties might be small organic molecule dyes, such as xanthene dyes, like fluorescein, or rhodamine dyes, coumarine dyes, cyanine dyes, pyrene dyes, oxazine dyes, pyridyl oxazole dyes, pyromethene dyes, acridine dyes, oxadiazole dyes, carbopyronine dyes, benzpyrylium dyes, fluorene dyes, or metallo-organic complexes, such as Ru, Eu, Pt complexes. Besides single molecule entities, clusters of small organic molecule dyes, fluorescent oligomers or fluorescent polymers, such as polyfluorene, can also be used as fluorescent moieties. Additionally, fluorescent moieties might be protein-based, such as phycobiliproteins, nanoparticles, such as quantum dots, upconverting nanoparticles, gold nanoparticles, dyed polymer nanoparticles.

The fluorescent moiety X can be covalently coupled to the linker unit L. Methods for covalently conjugation are known by persons skilled in the art. A direct reaction of an activated group either on the fluorescent moiety X or on the linker unit L with a functional group on either the linker unit L or on the fluorescent moiety X or via a heterobifunctional linker molecule, which is firstly reacted with one and secondly reacted with the other binding partner is possible.

For example, fluorescent dyes are available with groups reactive towards amino groups or thiol groups, such as active esters which react with amino groups on the linker unit, for instance N-hydroxysuccinimide esters (NHS), sulfodichlorophenyl esters (SDP), tetrafluorophenyl esters (TFP), and pentafluorophenyl esters (PFP), or Michael acceptors or haloacetyl groups, which react with thiol groups on the linker unit, for instance maleimide groups, iodoacetamide groups, and bromomaleimide groups. A large number of heterobifunctional compounds are available for linking to entities. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-y-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, succinimidyl-[(N-maleimidopropionamido) polyethyleneglycol] esters (NHS-PEG-MAL), and succinimidyl 4-[N-maleimidomethyl]cyclohexane-1-carboxylate. A preferred linking group is 3-(2-pyridyldithio)propionic acid N-hydroxysuccinimide ester (SPDP), or 4-(N-maleimidomethyl)-cyclohexane-1-carboxylic acid N-hydroxysuccinimide ester (SMCC) with a reactive sulfhydryl group on the fluorescent moiety and a reactive amino group on the linker unit.

The conjugate used in the method of the invention may comprise 1 to 100, preferable 2-30 fluorescent moieties X.

### Antigen Recognizing Moiety Y

The term “antigen recognizing moiety Y” refers to any kind of molecule which binds against the target moieties expressed on the biological specimens, like antigens expressed intracellular or extracellular on cells. The term “antigen recognizing moiety Y” relates especially to an antibody, a fragmented antibody, a fragmented antibody derivative, peptide/MHC-complexes targeting TCR molecules, cell adhesion receptor molecules, receptors for costimulatory molecules or artificial engineered binding molecules, peptides, lectins or aptamers, RNA, DNA, oligonucleotides and analogues thereof.

Fragmented antibody derivatives, are for example Fab, Fab′, F(ab′)2, sdAb, scFv, di-scFv, nanobodies. Such fragmented antibody derivatives may be synthesized by recombinant procedures including covalent and non-covalent conjugates containing these kind of molecules.

The conjugate used in the method of the invention may comprise 1 to 100, preferable 1 to 20 antigen recognizing moieties Y. The interaction of the antigen recognizing moiety with the target moiety can be of high or low affinity. Binding interactions of a single low-affinity antigen recognizing moiety is too low to provide a stable bond with the antigen. Low-affinity antigen recognizing moieties can be multimerized by conjugation to the enzymatically degradable spacer P to furnish high avidity.

Preferable, the term “Antigen recognizing moiety Y” refers to an antibody or Fab directed against antigen expressed by the biological specimens (target cells) intracellular, like IL2, FoxP3, CD154, or extracellular, like CD3, CD14, CD4, CD8, CD25, CD34, CD56, and CD133.

The antigen recognizing moieties Y, especially antibodies, can be coupled to the spacer P through side chain amino or sulfhydryl groups. In some cases, the glyosidic side chain of the antibody can be oxidized by periodate resulting in aldehyde functional groups.

The antigen recognizing moiety Y can be covalently or non-covalently coupled to the spacer P. Methods for covalent or non-covalent conjugation are known by persons skilled in the art and the same as mentioned for conjugation of the fluorescent moiety X.

### Enzymatically Degradable Spacer P

The enzymatically degradable spacer P can be any molecule which can be cleaved by a specific enzyme like a hydrolase. Suitable as enzymatically degradable spacer P are, for example, polysaccharides, proteins, peptides, depsipeptides, polyesters, nucleic acids, and derivatives thereof.

Suitable polysaccharides are, for example, dextrans, pullulans, inulins, amylose, cellulose, hemicelluloses, such as xylan or glucomannan, pectin, chitosan, or chitin, which may be derivatized to provide functional groups for covalent or non-covalent binding of the linker L and the antigen recognizing moiety Y. A variety of such modifications are known in the art, for example, imidazolyl carbamate groups may be introduced by reacting the polysaccharide with N,N′-carbonyl diimidazole. Subsequently amino groups may be introduced by reacting said imidazolyl carbamate groups with hexane diamine. Polysaccharides may also be oxidized using periodate to provide aldehyde groups or with N,N′-dicyclohexylcarbodiimide and dimethylsulfoxide to provide ketone groups. Aldehyde or ketone functional groups can be reacted subsequently preferably under conditions of reductive amination either with diamines to provide amino groups or directly with amino substituents on a proteinaceous binding moiety. Carboxymethyl groups may be introduced by treating the polysaccharide with chloroacetic acid. Activating the carboxy groups with methods known in the art which yield activated esters such N-hydroxysuccinimid ester or tetrafluorophenyl ester allows for reaction with amino groups either of a diamine to provide amino groups or directly with an amino group of a proteinaceous binding moiety. It is generally possible to introduce functional group bearing alkyl groups by treating polysaccharides with halogen compounds under alkaline conditions. For example, allyl groups can be introduced by using allyl bromide. Allyl groups can further be used in a thiol-ene reaction with thiol bearing compounds such as cysteamine to introduce amino groups or directly with a proteinaceous binding moiety with thiol groups liberated by reduction of disulfide bonds or introduced by thiolation for instance with 2-iminothiolane.

Proteins, peptides, and depsipeptides used as enzymatically degradable spacer P can be functionalized via side chain functional groups of amino acids to attach to linker L and antigen recognizing moiety Y. Side chains functional groups suitable for modification are for instance amino groups provided by lysine or thiol groups provided by cysteine after reduction of disulfide bridges.

Polyesters and polyesteramides used as enzymatically degradable spacer P can either be synthesized with co-monomers, which provide side chain functionality or be subsequently functionalized. In the case of branched polyesters functionalization can be via the carboxyl or hydroxyl end groups. Post polymerization functionalization of the polymer chain can be, for example, via addition to unsaturated bonds, i.e. thiolene reactions or azide-alkine reactions, or via introduction of functional groups by radical reactions.

Nucleic acids used as enzymatically degradable spacer P are preferably synthesized with functional groups at the 3′ and 5′ termini suitable for attachment of the binding moiety B and antigen recognizing moiety A. Suitable phosphoramidite building blocks for nucleic acid synthesis providing for instance amino or thiol functionalities are known in the art.

The enzymatically degradable spacer P can be composed of more than one different enzymatically degradable units, which are degradable by the same or different enzyme.

### Linker L

The linker L is a polar hydrophilic oliogomer, comprising between 2 and 500 preferably between 4 and 30 repeating units of ethylene glycol.

The linker group L may be linear to allow for the attachment of a single fluorescent moiety X. The linker moiety might comprise a functional or activated group on each end of the oligomer to react directly or via prior reaction with a heterobifunctional crosslinker with an activated or functional group on the fluorescent moiety and with an activated or functional group on the enzymatically degradable spacer P. The methods and groups employed are the same as described for the covalent attachment of the fluorescent moiety X. Alternatively the fluorescent moiety X might already comprise a polyethylene glycol chain with an activated or functional group, which can be conjugated to the enzymatically degradable spacer P. In this case the polyethylene glycol chain serves as the linker L.

In a particular useful embodiment of the invention commercially available heterobifunctional polyethylene glycols can be reacted with an activated fluorescent moiety on one end and be activated on the other end for reaction with the enzymatically degradable spacer P.

In another embodiment, the linker group L may be branched to allow for the attachment of multiple fluorescent moieties. In this embodiment, the linker unit L comprises one ore more polyethylene glycol residues which are bound to at least one (like one to six) polyhydroxy branching units chosen from core unit selected from the group consisting of polyhydroxy compounds, polyamino compounds, polythio compounds. Preferred as core unit are for example glycerol with three hydroxyl groups as attachment point for 3 polyether residues via ether bonds, pentaerythritol with four hydroxyl group as attachment points for 3 to 4 polyether residues via ether bonds, dipentaerythritol with six hydroxyl groups as attachment points for 3 to 6 polyether branches via ether bonds, tripentaerythritol or hexaglycerol with eight hydroxyl groups as attachment points for 3 to 8 polyether branches via ether bonds. In this embodiment, the linker L comprises a sum of 3 to 500 ethylene glycol repeating units.

In a particular useful embodiment of the invention commercially available multi-arm polyethylene glycols (branched PEGs) serve as linkers which include a branching moiety and polyether branches. The ends of the arms of the branched PEGs are functionalized or activated to allow for covalent attachment of fluorescent moieties or enzymatically degradable spacer P as described before. Multi-arm polyethylene glycols are commercialized by, for example, Nanocs Inc. or NOF Corporation.

The linker L can be covalently or quasi-covalently coupled to the enzymatically degradable spacer P. Methods for covalent or quasi-covalent conjugation are known by persons skilled in the art and the same as mentioned for conjugation of the fluorescent moiety X. A quasi-covalent binding of the fluorescent moiety X to the linker unit L can be achieved with binding systems providing an equilibrium dissociation constant of 10-9 M, e.g., Biotin-Avidin binding interaction.

### Method of the Invention

A preferred embodiment of the method of the invention comprises step d), in which the enzymatically degradable spacer P is degraded by an enzyme, thereby cleaving the fluorescent moieties X from the labelled target moiety.

In another embodiment of step d), the enzymatically degradable spacer P is degraded by an enzyme, thereby cleaving the fluorescent moieties from X and the antigen recognizing moieties Y are cleaved from the labelled target moiety.

The term “enzymatically degrading spacer P, thereby cleaving the fluorescent moiety X from the conjugate” means that covalent bonds of the fragment (XₒL)ₙ-P-Yₘ are cleaved by degrading spacer P in a way that at least the fluorescent moiety X and linker unit L are removed from the target moiety.

In a variant of the invention, the enzymatically degradable spacer P is degraded by an enzyme, thereby cleaving both the fluorescent moieties from X and the antigen recognizing moieties Y from the labelled target moiety. This variant will initiated by using either low-affinity antigen recognizing moieties like FABs and/or for m>1, like 2-5.

The process of the invention may be performed in one or more sequences of the steps a) to d). After each sequence, the fluorescent moiety and linker L and optionally the antigen recognizing moiety is released (removed) from the target moiety. Especially when the biological specimens are living cells which shall be further processed, the method of the invention has the advantage of providing unlabelled cells.

After and/or before each step a)-d) one or more washing steps can be performed to remove unwanted material like unbound conjugate (I) or released parts of the conjugate like fluorescent moiety X or antigen recognizing moiety Y or reagents used for disruption. The term “washing” means that the sample of biological specimen is separated from the environmental buffer by a suitable procedure, e.g., sedimentation, centrifugation, draining or filtration. Before this separation washing buffer can be added and optionally incubated for a period of time. After this separation, the sample can be filled or resuspended again with buffer.

The method of the invention provides a high flexibility for the specific labeling with the conjugate and release of the conjugate providing a plurality of different detection strategies.

Any step can be monitored qualitatively or quantitatively according to the fluorescent moieties X used or by other applicable quantitative or qualitative methods known by persons skilled in the art, e.g., by visual counting. This can be useful to determine the efficiency of the individual steps provided by the method of the invention.

Such methods for labeling are known by persons skilled in the art, like utilizing non-degradable conjugates according to general formula (III) to (VI) as explained in the following.

### Step a)

In step a) of the method, at least one conjugate with the general formula (I) is provided. In order to detect different target moieties or the same target moiety by different detection moieties, different conjugates having the general formula (I) can be provided, wherein the conjugates and its components, Y, P, L, X, o, n, m, have the same meaning, but can be the same or different kind and/or amount of antigen recognizing moiety Y and/or linker unit L and/or enzymatically degradable spacer P and/or fluorescent moiety X. In further embodiments of the method, it is possible to label the sample of biological specimen with enzymatically degradable conjugates not comprising the linker L.

In one of these embodiments, at least one conjugate having the general formula II is provided

(X)ₙ-P-Yₘ (II)
with Y: antigen recognizing moiety recognizing the target moiety,
   P: enzymatically degradable spacer,
   X: fluorescent moiety,
   n, m: integer between 1 and 100,
wherein X and Y are covalently bound to the enzymatically degradable spacer P and contacting the sample of biological specimens with the conjugate accoding to formula (II), thereby labeling the target moiety recognized by the antigen recognizing moiety Y.

It is furthermore possible to provide in addition to conjugates with the general formula (I) or (II) conjugates which do not comprise an enzymatically degradable Spacer P and will survive the optional cleaving step d). Such conjugates can be used to label the sample of biological specimen in or after any of the steps a)-d) for qualitatively or quantitatively monitoring.

Such further conjugates may have the general formulas (III) and (IV) (Xₒ-L)ₙ-P′-Yₘ (III) and/or or Xₙ-P′-Yₘ (IV); with Y, L, X, n, m having the same chemical meaning as in formula (I) but wherein P′ is a spacer which is not enzymatically degradable. X, Xo-L, P′ and Y can be covalently or non-covalently bound.

Further, at least one conjugate with the general formulas (V) and (VI) (Xₒ-L)ₙ-Yₘ (V) and/or Xₙ-Yₘ (VI); wherein Y, X, n, m have the same meaning as in formula (I) can be provided. X, Xₒ-L and Y can be covalently or non-covalently bound to each other.

The method may use a variety of combinations of conjugates. For example, a conjugate may comprise antibodies specific for two different epitopes, like two different anti-CD34 antibodies. Different antigens may be addressed with different conjugates comprising different antibodies, for example, anti-CD4 and anti-CD8 for differentiation between two distinct T-cell-populations or anti-CD4 and anti-CD25 for determination of different cell subpopulations like regulatory T-cells.

### Step b)

In step b), the target moiety of the sample of biological specimens is labelled with the conjugate according to formula (I) to (VI)

In a variant of the invention the contacting with more than one conjugate of the general formula (I) can proceed simultaneously or subsequently in more than one step b).

Furthermore, conjugates not recognized by a target moiety can be removed by washing for example with buffer before the target moiety labeled with the conjugate is detected or isolated in step c) or before a next contacting step b).

In a variant of the invention, it is possible to perform multiple steps b). In addition to conjugates according to formula (I) the step b) can compromise at least one conjugate of the general formula (II)-(VI) which can be incubated simultaneously or subsequently.

Conditions during incubation are known by persons skilled in the art and may be empirically optimized in terms of time, temperature, pH, etc. Usually incubation time is up to 1h, more usually up to 30 min and preferred up to 15 min. Temperature is usually 4-37° C., more usually less than 37° C.

### Step c)

The method and equipment to detect the target moiety labeled with the conjugate is determined by the fluorescent moiety X.

Targets labeled with the conjugate are detected by exciting the fluorescent moiety X and analyzing the resulting fluorescence signal. The wavelength of the excitation is usually selected according to the absorption maximum of the fluorescent moiety X and provided by LASER or LED sources as known in the art. If several different fluorescent moieties X are used for multiple color/parameter detection, care should be taken to select fluorescent moieties having not overlapping absorption and emission spectra, at least not overlapping absorption and emission maxima. The targets may be detected, e.g., under a fluorescence microscope, in a flow cytometer, a spectrofluorometer, or a fluorescence scanner. Light emitted by chemoluminescence can be detected by similar instrumentation omitting the excitation.

The method of the invention may be utilized not only for detecting target moieties, i.e., target cells expressing such target moieties, but also for isolating the target cells from a sample of biological specimens according to the fluorescent moiety X. In the method of the invention the term “detection” encompasses “isolation”.

For example, the detection of a target moiety by fluorescence may be used to trigger an appropriate separation process by optical means, electrostatic forces, piezoelectric forces, mechanical separation or acoustic means.

In one variant of the invention, suitable for such separations according to a fluorescence signal are especially flow sorters, e.g., FACS or TYTO or MEMS-based cell sorter systems, for example as disclosed in EP14187215.0 or EP14187214.3.

In further variants of the invention it is possible to combine at least one detection and/or isolation step c) simultaneous or in subsequent steps.

Furthermore, during or after isolation of the target moieties contaminating non-labelled moieties of the sample of biological specimen can be removed by washing for example with buffer.

### Step d)

After detection and/or isolation of the target moiety in step c) in step d) the spacer P is enzymatically degraded thereby cleaving at least the fluorescent moiety X, the linker unit L from the conjugate.

Depending on the antigen recognizing moiety Y, when the spacer P is enzymatically cleaved, the low-affinity antigen recognizing moieties will be monomerized and may dissociate which results in a complete removal of the fluorescent moiety X, the linker unit L, the spacer P, and the antigen recognizing moiety Y. High-affinity antigen recognizing moieties provide a stable bond which results in a removal of the fluorescent moiety X, the linker unit L and the spacer P.

In a variant of the invention, step d) can be performed outside the detection system, e.g., in a solution of the target moiety in a tube.

In another variant, the enzymatically degradation can be implemented in the detection setup. For example, the disruption may take place during the detection of the signal, e.g., during fluorescence microscopy, cytometry or photometry. The reduction of the detection signal might therefore be monitored in real time.

Optionally after disruption in d) there can be another step c) with detecting or isolating the target moiety.

The fluorescent moiety X and linker unit L and/or the enzymatically degraded spacer P and/or antigen recognizing moiety Y and/or residual target moieties still labelled with the conjugate (I) or non-cleaved parts of conjugate (I) and/or the reagent used for enzymatically degradation in c) can be separated from the sample by, e.g., washing or utilizing the methods described in step c).

Those one or more optionally detection and/or isolation steps provide a possibility to separate the released target moiety or determine the efficiency of the disruption step d).

Another variant of the invention comprises the elimination of a fluorescence emission by a combination of enzymatic degradation and oxidative bleaching. The necessary chemicals for bleaching are known from the above-mentioned publications on “Multi Epitope Ligand Cartography”, “Chip-based Cytometry” or “Multioymx” technologies.

### Enzymes for Degrading Spacer P

The enzymatically degradable spacer P is degraded by the addition of an appropriate enzyme. The choice of enzyme as release reagent is determined by the chemical nature of the enzymatically degradable spacer P and can be one or a mixture of different enzymes.

Enzymes are preferably hydrolases, but lyases or reductases are also possible. Preferable enzymes may be is selected from the group consisting of glycosidases, dextranases, pullulanases, amylases, inulinases, cellulases, hemicellulases, pectinases, chitosanases, chitinases, proteinases, esterases, lipases, and nucleases.

For example, if the spacer P is a polysaccharide, glycosidases (EC 3.2.1) are most suitable as release agents. Preferred are glycosidases that recognize specific glycosidic structures, e.g., dextranase (EC3.2.1.11), which cleaves at the α(1->6) linkage of dextrans, pullulanases, which cleave either α(1->6) linkages (EC 3.2.1.142) or α(1->6) and α(1->4) linkages (EC 3.2.1.41) of pullulans, neopullulanase (EC 3.2.1.135), and isopullulanase (EC 3.2.1.57), which cleave α(1->4) linkages in pullulans. α-Amylase (EC 3.2.1.1), and maltogenic amylase (EC 3.2.1.133), which cleave α(1->4) linkages in amylose, inulinase (EC 3.2.1.7), which cleaves β(2->1) fructosidic linkages in inulin, cellulase (EC 3.2.1.4), which cleaves at the β(1->4) linkage of cellulose, xylanase (EC 3.2.1.8), which cleaves at the β(1->4) linkages of xylan, pectinases such as endo-pectin lyase (EC 4.2.2.10), which cleaves eliminative at the α(1->4) D-galacturonan methyl ester linkages, or polygalacturonase (EC 3.2.1.15), which cleaves at the α(1->4) D-galactosiduronic linkages of pectin, chitosanase (EC 3.2.1.132), which cleaves at the β(1->4) linkages of chitosan and endo-chitinase (EC 3.2.1.14) for cleaving of chitin.

Proteins and peptides may be cleaved by proteinases, which need to be sequence specific to avoid degradation of target structures on cells. Sequence specific proteases are for instance TEV protease (EC 3.4.22.44), which is a cysteine protease cleaving at the sequence ENLYFQ\S, enteropeptidase (EC 3.4.21.9), which is a serine protease cleaving after the sequence DDDDK, factor Xa (EC 3.4.21.6), which is a serine endopeptidase cleaving after the sequences IEGR or IDGR, or HRV3C protease (EC3.4.22.28), which is a cysteine protease cleaving at the sequence LEVLFQ\GP.

Depsipeptides, which are peptides containing ester bonds in the peptide backbone, or polyesters may be cleaved by esterases, such as porcine liver esterase (EC 3.1.1.1) or porcine pancreatic lipase (EC 3.1.1.3). Nucleic acids may be cleaved by endonucleases, which can be sequence specific, such as restriction enzymes (EC 3.1.21.3, EC 3.1.21.4, EC 3.1.21.5), such as EcoRI, HindII or BamHI or more general such as DNAse I (EC 3.1.21.1), which cleaves phosphodiester linkages adjacent to a pyrimidine.

The amount of enzyme added needs to be sufficient to degrade substantially the spacer in the desired period of time. Usually the efficiency is at least about 80%, more usually at least about 95%, preferably at least about 99%. The conditions for release may be empirically optimized in terms of temperature, pH, presence of metal cofactors, reducing agents, etc. The degradation will usually be completed in at least about 15 minutes, more usually at least about 10 minutes, and will usually not be longer than about 2 h.

It is not necessary to degrade spacer P entirely. For the method of the invention, it is necessary to degrade spacer P as much that the fluorescent moiety X or the fluorescent moiety X and antigen recognizing moiety Y can be removed from the labeled target moiety by washing or dissociation.

### Sequences of Steps a) to d)

The method of the invention is especially useful for detection and/or isolation of specific target moieties from complex mixtures and may be performed in one or more sequences of the steps a) to d). After each sequence, the fluorescent moiety and optionally the antigen recognizing moiety Y is released (removed) from the target moiety. Furthermore, sequences with combinations of any of the steps a) to d) are possible. Sequences can be stopped at any of the steps a) to d). Additional washing steps can be implemented.

In a variant of the invention, at least two conjugates are provided simultaneously or in subsequent staining sequences, wherein each antigen recognizing moiety Y recognizes different antigens. In a further variant of the invention, at least two conjugates are provided simultaneously or in subsequent staining sequences, wherein each conjugate comprises a different fluorescent moiety X. In an alternative variant, at least two conjugates can be provided to the sample simultaneously or in subsequent staining sequences, wherein each conjugate comprises a different enzymatically degradable spacer P which is cleaved by different enzymes. In all cases, the labeled target moieties can be detected simultaneously or sequentially. Sequential detection may involve simultaneous enzymatically degrading of the spacer molecules P or subsequent enzymatically degrading of the spacer molecules P with optionally intermediate removing (washing) of the non-bonded moieties.

### Embodiments of Sequences of Steps a) to d)

The method of the invention can be performed in the following embodiments:

In all variants and embodiments, the conjugate of the general formula (I) may be used in mixture and/or if used in different sequences in combination with one or more of the conjugates according to general formula (II), (III), (IV), (V) and (VI).

Embodiment A of the invention is characterized in that steps a) to d) are performed in at least one sequence wherein in each sequence one conjugate of the general formula (I) or (II) is used. In this embodiment in at least one sequences the sample of biological specimen is contacted in step b) with one conjugate, the detection in performed in step c) and the conjugate in cleaved in step d). Therefore, embodiment A includes single or multiple cycles using one conjugate. In each cycle X, L, P, Y and o, n, m of the conjugates used can be the same or different kind or amount of antigen recognizing moiety Y and/or linker unit L and/or fluorescent moiety X and/or enzymatically degradable spacer P.

An example of this variant for a single cycle with a single conjugate is the isolation by fluorescent based flow sorting of a cell population defined by the conjugate out of a sample of biological specimen wherein the fluorescent label is eliminated after sorting providing different downstream applications.

An example for this variant for multiple cycles with single conjugates is the sequential detection of different target moieties by using different antigen recognizing moieties and the same fluorescent moiety in cycles of labeling-detection-elimination, which enables high multiplexing potential for, e.g., protein mapping on cells by microscopy. Another example is the isolation by fluorescent based flow sorting of cell subpopulations out of a sample of biological specimen in sequential sorting cycles using the same fluorescent moiety. In a further example the same target moiety can be addressed in a first cycle with a conjugate having a fluorescent moiety suitable for flow sorting purposes and after release of this fluorescent moiety the target moiety can be readdressed by a conjugate having another fluorescent moiety especially suitable for analysis by fluorescent microscopy.

Embodiment B of the invention is characterized in that steps a) to d) are performed in at least one sequence wherein in each sequence at least a first and a second conjugate of the general formula (I) or (II) are used. In this embodiment in at least one sequence the sample of biological specimen is contacted in simultaneous or subsequent steps b) with at least a first and a second conjugate, the detection in performed in simultaneous or subsequent steps c) and the conjugate in cleaved in subsequent or simultaneous steps d). Therefore, embodiment B includes single or multiple cycles using multiple conjugates. In each cycle X, L, P, Y and o, n, m of the conjugates used can be the same or different kind or amount of antigen recognizing moiety Y and/or linker unit L and/or fluorescent moiety X and/or enzymatically degradable spacer P.

An example for this variant for a single cycle with multiple conjugates is the simultaneous labeling with different conjugates which enables differentiation of different cell subpopulations by flow cytometry analysis and isolation of a defined subpopulation by fluorescent based flow sorting wherein the fluorescent label is eliminated after sorting providing different downstream applications.

An example for this variant for multiple cycles with multiple conjugates is the sequential detection of different target moieties by using different antigen recognizing moieties and different fluorescent moieties in cycles of labeling-detection-elimination, which enables even higher multiplexing potential.

Embodiment C of the invention is characterized in that steps a) to c) are performed in at least two sequences wherein in each sequence one conjugate of the general formula (I) or (II) is used and step d) is performed afterwards. In this embodiment in at least two sequences the sample of biological specimen is contacted in step b) with one conjugate and the detection in performed in step c). After a least two of those sequences the conjugates are cleaved in subsequent or simultaneous step d). Therefore, embodiment C includes single or multiple cycles a)-c) using one conjugate and a step d). In each cycle X, L, P, Y and o, n, m of the conjugates used can be the same or different kind or amount of antigen recognizing moiety Y and/or linker unit L and/or fluorescent moiety X and/or enzymatically degradable spacer P.

Embodiment D of the invention is characterized in that steps a) to c) are performed in at least two sequences wherein in each sequence at least a first and a second conjugate of the general formula (I) or (II) are used and step d) is performed afterwards. In this embodiment in at least two sequences the sample of biological specimen is contacted in simultaneous or subsequent steps b) with at least a first and a second conjugate and the detection in performed in simultaneous or subsequent steps c). After a least two of those sequences the conjugates are cleaved in subsequent or simultaneous step d). Therefore, embodiment D includes single or multiple cycles a)-c) using multiple conjugates and a step d). In each cycle the conjugates used can be the same or different X, L, P, Y and o, n, m can be the same or different amount of antigen recognizing moiety Y and/or linker unit L and/or enzymatically degradable spacer P and/or fluorescent moiety X.

Embodiment E of the invention is characterized in that steps a) to b) are performed in at least two sequences wherein in each sequence one conjugate of the general formula (I) or (II) is used and step c) and d) is performed afterwards. In this embodiment in at least two sequences the sample of biological specimen is contacted in step b) with one conjugate. After a least two of those sequences the detection in performed in subsequent or simultaneous step c) and the conjugates are cleaved in subsequent or simultaneous step d). Therefore, embodiment E includes single or multiple cycles a)-b) using one conjugate and step c) and step d). In each cycle X, L, P, Y and o, n, m of the conjugates used can be the same or different kind or amount of antigen recognizing moiety Y and/or linker unit L and/or fluorescent moiety X and/or enzymatically degradable spacer P.

Embodiment F of the invention is characterized in that steps a) to b) are performed in at least two sequences wherein in each sequence at least a first and a second conjugate of the general formula (I) or (II) are used and step c) and d) is performed afterwards. In this embodiment in at least two sequences the sample of biological specimen is contacted in simultaneous or subsequent steps b) with at least a first and a second conjugate. After a least two of those sequences the detection in performed in simultaneous or subsequent steps c) and the conjugates are cleaved in subsequent or simultaneous step d). Therefore, embodiment D includes single or multiple cycles a)-b) using multiple conjugates and step c) and step d). In each cycle X, L, P, Y and o, n, m of the conjugates used can be the same or different kind or amount of antigen recognizing moiety Y and/or linker unit L and/or fluorescent moiety X and/or enzymatically degradable spacer P.

An example for Embodiment C to F is the step by step analysis of individual target moieties in a sample of biological specimen with sequential overlaying of signals wherein after a certain amount of cycles the signals can be completely or just partially eliminated enabling further cycles. Compared to embodiments A and B those embodiments provide a higher flexibility.

Embodiment G of the invention is characterized in that steps a) to d) are performed in at least two interlaced sequences wherein in each sequence one conjugate of the general formula (I) or (II) is used. In this embodiment in at least two sequences the sample of biological specimen is contacted in step b) with one conjugate, the detection in performed in step c) and the conjugate is cleaved in step d) wherein step d) of the first cycle and step b) of the second cycle are combined in one simultaneous step. Therefore, embodiment G includes interlaced multiple cycles using each cycle one conjugate. In each cycle X, L, Y and o, n, m of the conjugates used can be the same or different kind or amount of antigen recognizing moiety Y and/or linker unit L and/or fluorescent moiety X. At least every second cycle the enzymatically degradable spacer P is of different kind.

Embodiment H of the invention is characterized in that steps a) to d) are performed in at least two interlaced sequences wherein in each sequence at least a first and a second conjugate of the general formula (I) or (II) are used. In this embodiment in at least two sequences the sample of biological specimen is contacted in simultaneous or subsequent steps b) with at least a first and a second conjugate, the detection in performed in simultaneous or subsequent steps c) and the conjugate in cleaved in subsequent or simultaneous steps d) wherein step d) of the first cycle and step b) of the second cycle are combined in one simultaneous step. Therefore, embodiment G includes interlaced multiple cycles using multiple conjugates. In each cycle X, L, Y and o, n, m of the conjugates used can be the same or different kind or amount of antigen recognizing moiety Y and/or linker unit L and/or fluorescent moiety X. At least every second cycle the enzymatically degradable spacer P is of different kind.

Compared to embodiment A and B the process according to embodiment G or H provides a reduction of time for multiple cycles of labelling, detection and enzymatically degradation of spacer P. A requirement of these embodiments is the use of at least two different enzymatically degradable spacer P and accordingly different enzymes as release reagent which can be used orthogonal to each other.

### Use of the Method

The method of the invention can be used for various applications in research, diagnostics and cell therapy.

In a first use of the invention, biological specimens like cells are detected or isolated for counting purposes i.e. to establish the amount of cells from a sample having a certain set of antigens recognized by the antigen recognizing moieties of the conjugate.

In a second use, one or more populations of biological specimens are separated for purification of target cells. Those isolated purified cells can be used in a plurality of downstream applications like molecular diagnostics, cell cultivation, or immunotherapy.

In other uses of the invention, the location of the target moieties like antigens on the biological specimens recognized by the antigen recognizing moieties of the conjugate is determined. Advanced imaging methods are known as “Multi Epitope Ligand Cartography”, “Chip-based Cytometry” or “Multioymx” and are described, for example, in EP 0810428, EP1181525, EP 1136822 or EP1224472. In this technology, samples of biological specimen are contacted in sequential cycles with antigen recognizing moieties coupled to a fluorescent moiety, the location of the antigen is detected by the fluorescent moiety and the fluorescent moiety is afterwards eliminated. Therefore, subsequent cycle of labelling-detection-elimination with at least one fluorescent moiety provide the possibility to map protein networks, localize different cell types or the analysis of disease-related changes in the proteome.

### EXAMPLES

### Example 1—Conjugation of Dextran-PEG-Coumarin-Dye and Dextran-Coumarin-Dye and Determination of Fluorescence Quenching

To prepare conjugates according to the invention the small organic molecule dye, e.g., a coumarin-dye like Pacific Blue NHS-ester (available from Thermo Fisher Scientific) was dissolved in DMSO and carboxy-PEG-amine, e.g., CA(PEG)24 (available from Thermo Fisher Scientific) dissolved in DMSO, added. The reaction mixture was stirred for 2 h at room temperature. Afterwards the carboxy-PEG-coumarin-dye was activated by adding EDC and NHS (available, e.g. by Merck) over night at room temperature.

In the next step, dextran-fluorochrome-conjugates, according to the invention (Xₒ-L)ₙ-P and according to prior art (X)ₙ—P, were prepared by incubation of aminodextran (70 kDa) (available from Fina Biosolutions) at concentration of 10 mg/mL with the activated NHS-PEG-coumarin-dye, respectively the NHS-coumarin-dye like Pacific Blue, in different molar ratios of dextran: NHS-coumarin-dye=1:10 to 1:24. After 60 min incubation time at room temperature, the dextran-fluorochrome-conjugate was purified by size exclusion chromatography utilizing PBS/EDTA-buffer. The amount of conjugated coumarin-dye and degree of labeling (DOL) was determined by the absorbance at the specific wavelength of the fluorescent dye, for coumarin-dye 416 nm. The DOL was 4.1, 6.5 and 8.6 for dextran-PEG-coumarin-dye and 3.7, 5.0, 7.8 for dextran-coumarin-dye.

Dextran-PEG-coumarin-dye- and dextran-coumarin-dye-conjugates were diluted to the same concentration of dextran to determine the dependency of the fluorescence quenching on the degree of labeling. The absorbance at the specific wavelength of the fluorescent dye, for coumarin dye 416 nm, and the emission intensity after excitation at 416 nm was determined.

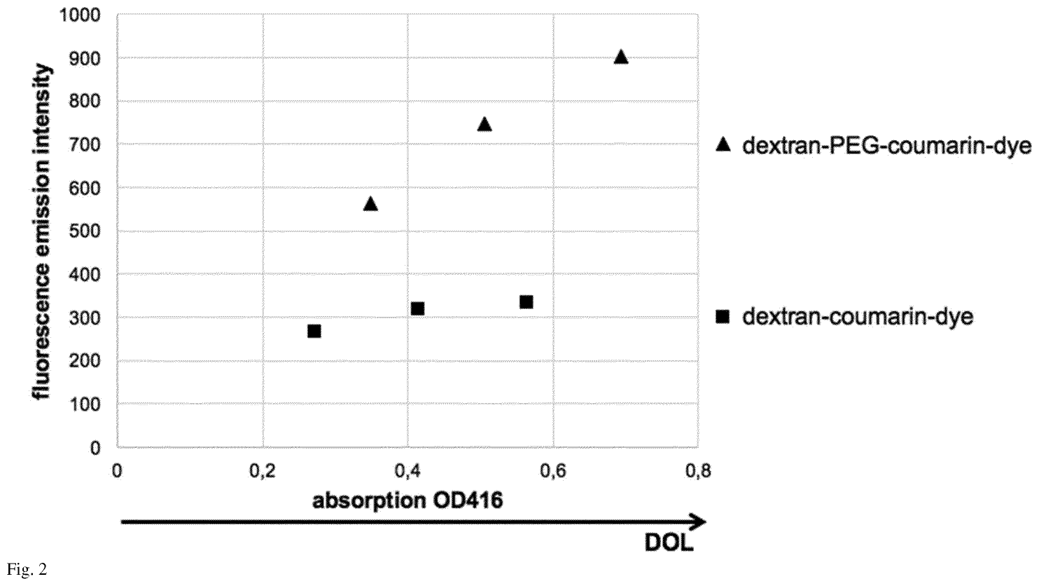

FIG. 2 shows exemplary the absorption and emission intensity of the dextran-PEG-coumarin-dye- and dextran-coumarin-dye-conjugates. For dextran-coumarin-dye the fluorescence emission intensity only minimal increases with increasing absorbance, respectively DOL, indicating the strong quenching of the fluorescence of the coumarin molecules on the dextran molecule. In contrast, for dextran-PEG-courmarin-dye the fluorescence emission intensity is higher at a comparable DOL. The intensity increases with increasing absorbance, respectively DOL, indicating that the PEG-linker prevents the quenching of the coumarin molecules.

### Example 2—Reversible Cell Surface Staining and Flow Cytometry Analysis with Fab-Dextran-Coumarin-Dye- and Fab-Dextran-PEG-Coumarin-Dye-Conjugates

To prepare antibody- or Fab-dextran-fluorochrome-conjugates, according to formula (I) (Xₒ-L)ₙ-P-Yₘ or formula (II) (X)ₙ-P-Yₘ, the dextran-PEG-coumarin-dye- and dextran-coumarin-dye-conjugates were activated by incubation with SMCC for 60 min at room temperature and purified by size exclusion chromatography utilizing PBS/EDTA-buffer. Antibody or Fab, e.g., anti-CD4, was reduced with 10 mM DTT in MES-buffer. After 90 min incubation time at room temperature, the antibody was purified by size exclusion chromatography utilizing PBS/EDTA-buffer. For the conjugation of the antibody- or Fab-dextran-fluorochrome-conjugate activated Fab or antibody was added to the activated dextran. After 60 min incubation time at room temperature, β-mercaptoethanol followed by N-ethylmaleimide were added sequentially with molar excess to block unreacted maleimide- or thiol-functional groups. The antibody- or Fab-dextran-fluorochrome-conjugate was purified by size exclusion chromatography utilizing PBS/EDTA-buffer. The concentrations of antibody or Fab and fluorescent moiety were determined by the absorbance at 280 nm and absorbance at the specific wavelength of the fluorescent dye.

Cell Surface Staining

PBMCs in PBS/EDTA/BSA-buffer were stained for 10 min at 4° C. with anti-CD4-Fab-dextran-coumarin-dye-conjugate DOL 5.0 or with anti-CD4-Fab-dextran-PEG-coumarin-dye-conjugate DOL 4.1, 6.5, and 8.6. The cells were washed with cold PBS/EDTA-BSA-buffer and analyzed by flow cytometry. For reversibility of the fluorescent labeling cells were incubated with dextranase for 10 min at 21° C., washed with PBS/EDTA-BSA-buffer and analyzed by flow cytometry.

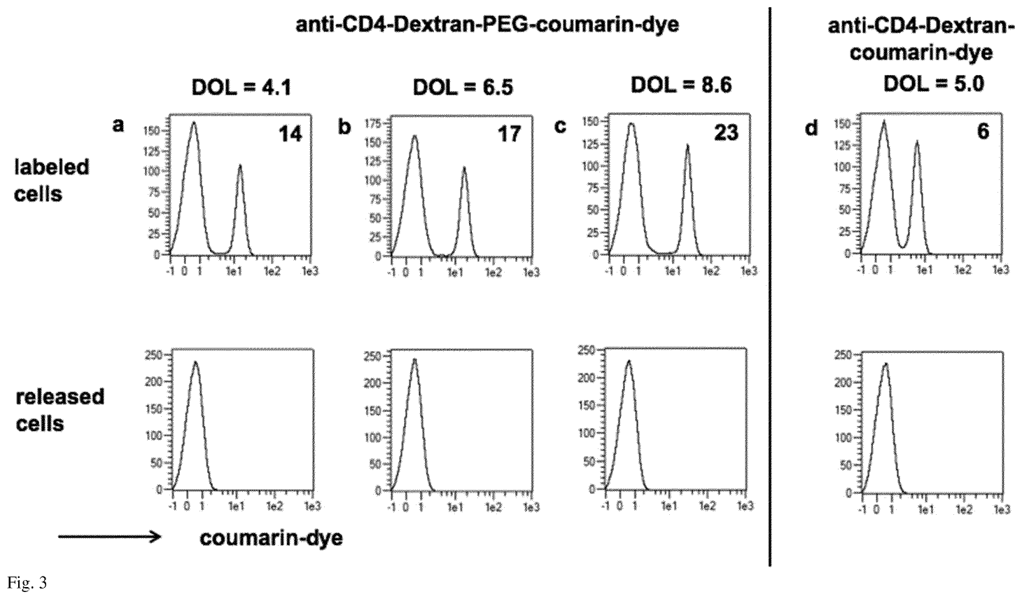

FIG. 3 shows exemplary histograms of the result of flow cytometry analysis of the single parameter labeling with the different anti-CD4-Fab-dextran-PEG-coumarin-dye (a-c) and anti-CD4-Fab-dextran-coumarin-dye-conjugates (d) (pregating on lymphocytes and exclusion of dead cells by propidium iodide, upper right: mean fluorescence intensity of CD4+ T-cell population). Depending on the DOL, cells stained with anti-CD4-Fab-dextran-PEG-coumarin-dye are 2.3-3.8-fold brighter as cells stained with the anti-CD4-dextran-coumarin-dye. After the addition of the dextran-degrading enzyme dextranase the remaining fluorescence intensity of the labeled CD4+ T-cell population is in the range of the detection limit.

1. A conjugate for labelling a target moiety on a cell, characterized with the general formula I
(Xo-L)n-P-Ym (1)
with Y: antigen recognizing moiety recognizing the target moiety,
P: enzymatically degradable spacer,
X: fluorescent moiety,
L: linker unit comprising one or more polyethyleneglycol residues n, m: integer between 1 and 100,
o: integer between 1 and 100
wherein L covalent hounds the fluorescent moiety X and the enzymatically degradable spacer P and Y is covalently bound to the enzymatically degradable spacer P and wherein the enzymatically degradable spacer P is selected from the group consisting of polysaccharides, polyesters, nucleic acids, and derivatives thereof.

2. The conjugate according to claim 1 characterized in that the linker unit L comprises one or more polyethylene glycol residues which are bound to at least one core unit selected from the group consisting of polyhydroxy compounds, polyamino compounds, polythio compounds.

3. The conjugate according to claim 1 characterized in that the linker unit L comprises one or more polyethyleneglycol residues with 2 to 500 repeating units of ethyleneglycol.

4. The conjugate according to claim 1, characterized in that antigen recognizing moiety Y is an antibody, a fragmented antibody, a fragmented antibody derivative, peptide/MHC-complexes targeting TCR molecules, cell adhesion receptor molecules, receptors for costimulatory molecules or artificial engineered binding molecules, peptides, lectins or aptamers, RNA, DNA, oligonucleotides and analogues thereof.

5. The conjugate according to claim 1, characterized in that the fluorescent moiety is selected from the group consisting of xanthene dyes, rhodamine dyes, coumarine dyes, cyanine dyes, pyrene dyes, oxazine dyes, pyridyl oxazole dyes, pyrromethene dyes, acridine dyes, oxadiazole dyes, carbopyronine dyes, benzopyrylium dyes, fluorene dyes, fluorescent oligomers or fluorescent polymers.

6. The conjugate according to claim 1, characterized in that the enzymatically degradable spacer P is further provided with at least one covalent hound linker unit L not hound to a fluorescent moiety X and/or with at least one covalent bound fluorescent moiety X not bound to a linker unit L according to general formula (X₀-L)ₙ-P(L)i(X)ₓ-Yₘ. wherein l and x are integer between 0 and 100.

7. A method for detecting a target moiety in a sample of biological specimen by:
a) providing at least one conjugate having the general formula I
(Xo-L)n-P-Ym (1)
with Y: antigen recognizing moiety recognizing the target moiety,
P: enzymatically degradable spacer,
X: fluorescent moiety,
L: linker unit comprising one or more polyethyleneglycol residues n, m: integer between 1 and 100,
o: integer between 1 and 100
wherein L covalent bounds the fluorescent moiety X and the enzymatically degradable spacer P and Y is covalently bound to the enzymatically degradable spacer P.
b) contacting the sample of biological specimens with the conjugate according to formula (I), thereby labeling the target moiety recognized by the antigen recognizing moiety Y; and
c) detecting the target moiety labelled with the conjugate with the fluorescent moiety X.

8. The method according to claim 7, characterized in that in step d), the enzymatically, degradable spacer P is degraded by an enzyme, thereby cleaving the fluorescent moieties X from the labelled target moiety.

9. The method according to claim 7, characterized in that in step d), the enzymatically degradable spacer P is degraded by an enzyme, thereby cleaving the fluorescent moieties from X and the antigen recognizing moieties Y from the labelled target moiety.

10. The method according claim 7 characterized in that the enzyme used for degrading the enzymatically degradable spacer P is selected from the group consisting of glycosidases, dextranases, pullulanases, amylases, inulinases, cellulases, hemicellulases, pectinases, chitosanases, chitinases, proteinases, esterases, lipases, and nucleases.

11. The method according to claim 7, characterized in further providing at least one conjugate having the general formula II
(X)n-P-Ym (II)
with Y: antigen recognizing moiety recognizing the target moiety,
P: enzymatically degradable spacer,
X: fluorescent moiety,
n, m: integer between 1 and 100,
wherein X and Y are covalently hound to the enzymatically degradable spacer 1 and contacting the sample of biological specimens with the conjugate according to formula (II), thereby labeling target moiety recognized by the antigen recognizing moiety Y.

12. The method according to claim 7 characterized in that the enzymatically degradable spacer P is further provided with at least one covalent bound linker unit L not bound to a fluorescent moiety X and/or with at least one covalent hound fluorescent moiety X not bound to a linker unit L according to general formula (X₀-L)ₙ-P(L)i(X)ₓ—Yₘ- wherein 1 and x are integer between 0 and 100.
